(19) United States
(12) Patent Application Publication
(10) Pub. No.: US 2023/0268032 A1
(43) Pub. Date: Aug. 24, 2023

(54) METHOD FOR GENERATING TRAINED MODEL, METHOD FOR DETERMINING BASE SEQUENCE OF BIOMOLECULE, AND BIOMOLECULE MEASUREMENT DEVICE

(71) Applicant: HITACHI HIGH-TECH CORPORATION, Minato-ku, Tokyo (JP)

(72) Inventors: Tatsuo NAKAGAWA, Tokyo (JP); Yusuke GOTO, Tokyo (JP); Rena AKAHORI, Tokyo (JP); Michiru FUJIOKA, Tokyo (JP)

(21) Appl. No.: 18/017,123

(22) PCT Filed: Jul. 31, 2020
(86) PCT No.: PCT/JP2020/029565

§ 371 Date: Jan. 20, 2023

(51) Int. Cl. G16B 40/10; G06N 3/045; G06N 3/08; G16B 40/20; G16B 30/00

(52) U.S. Cl. CPC G16B 40/10; G06N 3/045; G06N 3/08; G16B 40/20; G16B 30/00

(57) ABSTRACT

Provided is a method for generating a trained model for classifying blocking event data representing nanopore blocking events in a biomolecule measurement device. The method includes generating a first trained model by executing machine learning of a training model using first teacher data, the first teacher data includes teacher blocking event data and a teacher label, the teacher label indicates whether the teacher blocking event data is classified as Good data or bad data, and the first trained model is configured to classify the blocking event data into good data or bad data. In addition, a method for determining a base sequence a biomolecule and a biomolecule measurement device are provided.

### TECHNICAL FIELD

The present invention relates to a method for Generating a trained model, a method for determining a base sequence of biomolecule, and a biomolecule measurement device. For example, the present invention relates to a biopolymer analyzer that analyzes a base sequence of a biomolecule by a thin film in which a nano-sized pore is formed.

### BACKGROUND ART

In the field of DNA sequencers, attention is paid to a method for electrically directly measuring a base sequence of a biomolecule (in this case, DNA) without performing an elongation reaction or fluorescent labeling. Specifically, research and development of a nanopore DNA sequencing method have been actively promoted. This method is a method in which a DNA strand is directly measured without using a reagent to determine a base sequence.

In this nanopore DNA sequencing method, a base sequence is measured by measuring a blocking current generated when a DNA strand passes through a pore (hereinafter, referred to as “nanopore”.) formed in a thin film while blocking the pore. That is, since the blocking current changes with time depending on the difference in individual base species contained in the DNA strand, the base species can be sequentially identified by measuring the time series of the amount of the blocking current. In this method, the template DNA is not amplified by an enzyme, and a labeled substance such as a phosphor is not used. Therefore, high throughput, low running cost, and DNA decoding of long bases become possible.

In the nanopore DNA sequencing method, a device for biomolecule analysis used for analyzing DNA generally includes first and second liquid tanks filled with an electrolyte solution, a thin film partitioning the first and second liquid tanks, and first and second electrodes provided in the first and second liquid tanks. The device for biomolecule analysis can also be configured as an array device. The array device refers to a device including a plurality of sets of liquid chambers partitioned by thin films. For example, the first liquid tank is a common tank, and the second liquid tank is a plurality of individual tanks. In this case, an electrode is disposed in each of the common tank and the individual tanks.

In this configuration, when a voltage is applied between the first liquid tank and the second liquid tank, an ion current corresponding to the nanopore diameter flows through the nanopore. In addition, a potential gradient corresponding to the applied voltage is formed in the nanopore. If the biomolecule is introduced into the first liquid tank, the diffused biomolecule is sent to the second liquid tank via the nanopore according to the generated potential gradient. At this time, analysis of the inside of the biomolecule is performed according to the blocking rate of each nucleic acid blocking the nanopore. The biomolecule analyzer includes a measurement unit that measures a blocking signal (a signal representing an ion current flowing between electrodes provided in the device for biomolecule analysis), and acquires sequence information of the biomolecule based on a value of the measured blocking signal.

PTL 1 discloses the following classification analysis method. A particle passage detection signal is detected by a nanopore device according to passage of particles of a specimen through a through-hole. Based on a data group of the detected particle passage detection signal, a feature indicating a feature of a waveform shape of a pulsed signal corresponding to passage of a predetermined analyte is obtained. A classification analysis program based on machine learning is executed with the obtained feature as training data for machine learning and the feature obtained from the pulsed signal of the data to be analyzed as a variable. In this way, by performing classification analysis on a predetermined analyte in the data to be analyzed, the classification analysis of a particulate or molecular analyte can be performed with high accuracy.

In addition, PTL 2 discloses a biological sample analyzer including an accelerometer that detects vibration of an analyzer. By deleting or correcting the current value corresponding to vibration detection, the problem that the accuracy of base sequence decoding decreases due to environmental vibration is solved.

Further, PTL 3 discloses the following configuration. A control chain and a molecular motor are connected to a first end portion of the biomolecule. The control chain is bonded to a primer upstream thereof and has a spacer downstream thereof. While the transport control is performed, the control of a synthesis start point is appropriately performed.

In addition, NPL 1 discloses a configuration in which a reference current waveform of a target base sequence is generated from a database of base sequences and current values and compared with the measured current waveform to measure only the target current waveform.

### CITATION LIST

### Patent Literature

PTL 1: JP 2017-120257 A

PTL 2: JP 2019-27980 A

PTL 3: JP 2020-31557 A

### Non-patent Literature

NPL 1: Loose M, Malla S, Stout M., Real-time selective sequencing using nanopore technology., Nat

Methods. 2016;13(9):751-754. doi:10. 1038/nmeth. 3930

### SUMMARY OF INVENTION

### Technical Problem

One of the problems of nanopore DNA sequencers is the accuracy of sequencing.

It is required to read the base sequence of DNA that has passed through the nanopore with high accuracy. One of factors that hinder highly accurate sequencing is that signals which are not targets are mixed in the blocking signal of the nanopore. Specific examples thereof include a blocking event caused by impurities.

In a case of using the biomolecule disclosed in PTL 3, a signal to be read as a target is a signal in which a control chain and a molecular motor are connected to an end portion of DNA, and bonded to a primer on the upstream side thereof. However, in practice, not only such DNA to which the molecular motor and the primer are connected, but also DNA to which the molecular motor is not connected and DNA to which the primer is not connected may be electrophoresed in the nanopore and observed as a blocking event. Alternatively, even if the molecular motor is connected to DNA, the signal may become unstable due to a decrease in the activity of the molecular motor. In addition, only a polymerase or helicase that is a molecular motor can be observed as a blocking signal. Alternatively, it is also conceivable that a blocking signal is observed due to other particles or impurities contained in a solution. Since these signals that are not targets are mixed, when base calling (decoding a base sequence on the basis of a blocking signal) is performed, it is decoded as an incorrect base sequence, and the accuracy is degraded.

The present invention has been made in view of such a problem, and an object thereof is to improve the accuracy of sequencing by extracting a signal to be measured from blocking events in which signals not to be measured are mixed.

The foregoing and other objects and novel features of the present invention will become apparent from the description of the present specification and the accompanying drawings.

### Solution to Problem

As an example of a method for generating a trained model according to the present invention, there is provided a method for generating a trained model for classifying blocking event data representing a nanopore blocking event in a big molecule measurement device, the method including:

generating a first trained model by executing machine learning of a training model using first teacher data, wherein

the first teacher data includes teacher blocking event data and a teacher label, and the teacher label indicates whether the teacher blocking event data is classified as good data or bad data, and

the first trained model is configured to classify the blocking event data into good data or bad data.

Further, according to the present invention, a method for determining a base sequence of a biomolecule includes:

inputting blocking event data representing a blocking event of a nanopore in a biomolecule measurement device to a first trained model generated using the method described above;

classifying the blocking event data into good data or bad data by the first trained model; and

determining a base sequence of a biomolecule based on the blocking event data classified as good data.

Further, according to the present invention, a biomolecule measurement device includes:

a first liquid tank;

a second liquid tank;

a thin film on which nanopores are formed, the thin film being disposed between the first liquid tank and the second liquid tank;

a first electrode provided in the first liquid tank;

a second electrode provided in the second liquid

an ammeter that measures a current value flowing between the first electrode and the second electrode;

an extraction device that extracts blocking event data based on the current value measured by the ammeter;

a storage device that stores the blocking event data;

the first trained model described above that classifies the blocking event data into good data or bad data; and

a base caller that determines a base sequence of a biomolecule based on the blocking event data classified as the good data.

### ADVANTAGEOUS EFFECTS OF INVENTION

As an example of the effect according to the invention, the accuracy of sequencing is improved.

### BRIEF DESCRIPTION OF DRAWINGS

FIG. 1 is a schematic view illustrating a configuration example of a biomolecule measurement device according to a first embodiment.

FIG. 2 is a flowchart illustrating an example of a data processing method according to the first embodiment.

FIG. 3 is a flowchart illustrating an example of a method for classifying blocking event data according to the first embodiment.

FIG. 4 is a flowchart illustrating an example of a training method for generating a first trained model constituting a classifier according to the first embodiment.

FIG. 5 is a diagram schematically illustrating an example of a training model according to the first embodiment and an example of machine learning processing thereof.

FIG. 6 is a diagram schematically illustrating a biomolecule measurement device according to a second embodiment.

FIG. 7 is a diagram schematically illustrating a biomolecule measurement device according to a third embodiment.

FIG. 8 is a flowchart illustrating an example a feedback method according to the third embodiment.

FIG. 9 is an example of a current waveform according to a fourth embodiment.

FIG. 10 is an enlarged view of a blocking event data of FIG. 9.

FIG. 11 is a diagram obtained by discretizing the blocking event data of FIG. 10.

FIG. 12 is a functional block diagram of a computer of FIG. 1.

### DESCRIPTION OF EMBODIMENTS

In each of the following embodiments, when necessary for the sake of convenience, the description will divided into a plurality of sections or embodiments, but unless otherwise specified, the sections or embodiments are not unrelated to each other, and one is in a relationship of some or all modifications, details, supplementary explanation, and the like of the other. In addition, in the following embodiments, when referring to the number of elements or the like (including number, numerical value, amount, range, and the like), the number is not limited to a specific number unless otherwise specified or except for a case of being obviously limited to the specific number in principle, and may be more than or less than the specific number.

Furthermore, in each of the following embodiments, it goes without saying that the constituent elements (including element steps and the like) are not necessarily essential unless otherwise specified or except for a case of being considered to be obviously essential in principle. Similarly, in each of the following embodiments, when referring to the shape, positional relationship, and the like of the components and the like, it is assumed to include those substantially approximate or similar to the shape and the like, and the like, unless otherwise specified or except for a case of being clearly considered not to be in principle. The same applies to the above numerical values and ranges.

In all the drawings for describing the respective embodiments, the same members are denoted by the same reference numerals, and repeated description thereof may be omitted.

Note that, although the drawings illustrate specific embodiments conforming to the principles of the present invention, these are for understanding the present invention and are not used to interpret the present invention in a limited manner. Deoxyribonucleic acid (DNA) is exemplified as a biomolecule to be analyzed, but the biomolecule is not limited to DNA, and may be nucleic acid such as ribonucleic acid (RNA).

The “nanopore” described in each example of the present specification is a small through hole provided in a thin film. It may be called a micropore. The nanopore has a diameter expressed in a nanometer, for example, and is conventionally referred to as “nanopore”, and the size is not particularly limited as long as the pore is available for measuring a blocking event in a biomolecule measurement device.

The nanopore penetrates the front and back of the thin film. The thin film is mainly formed of an inorganic material. The substrate or bead to which one end of a DNA fragment is fixed is mainly formed of an inorganic material. The material of the thin film, the substrate, or the bead can also include an organic substance, a polymer material, or the like.

### First Embodiment

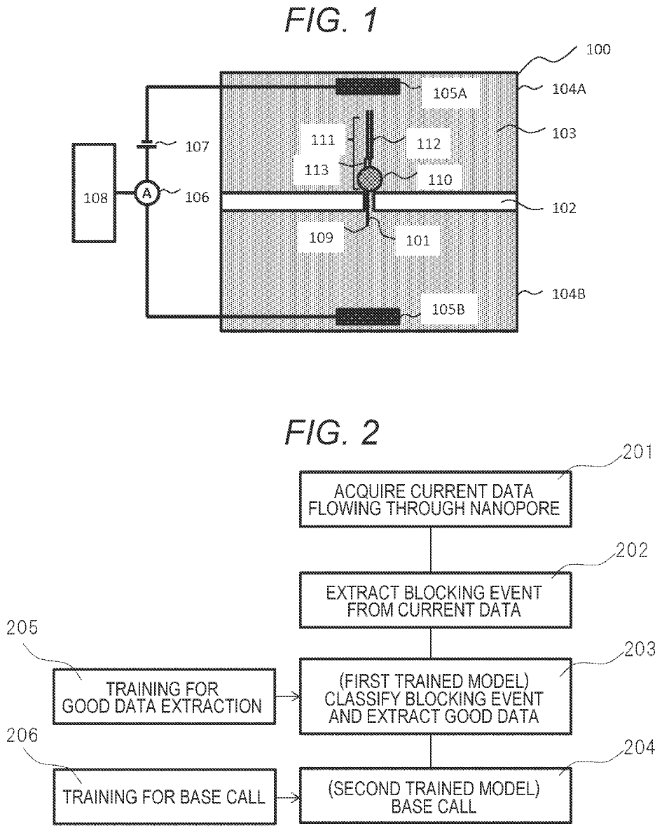

A method for generating a trained model, a method for determining a base sequence of a biomolecule, and a biomolecule measurement device according to a first embodiment of the present invention will be described with reference to FIGS. 1 to 5. FIG. 1 is a schematic view illustrating a configuration example of a biomolecule measurement device 100 according to the first embodiment. The biomolecule measurement device 100 is a device for biomolecule analysis that measures an ion current by a blocking current method.

The biomolecule measurement device 100 includes a liquid tank 104. The liquid tank 104 includes a first liquid tank 104A and a second liquid tank 104B. The biomolecule measurement device 100 includes a thin film 102. The thin film 102 is disposed between the first liquid tank 104A and the second liquid tank 104B.

The thin film 102 is formed of, for example, a solid material. A nanopore 101 is formed in the thin film 102. The nanopore 101 is a pore penetrating the thin film 102 between the first liquid tank 104A and the second liquid tank 104B. The thin film 102 contacts the first liquid tank 104A and the second liquid tank 104B to isolate them from each other at a portion other than the nanopore 101. According to such a configuration, is possible to accurately detect a current change due to a biomolecule.

In the device illustrated in FIG. 1, one thin film 102 has only one nanopore 101, but this is merely an example. It is also possible to form an array device by forming the plurality of nanopores 101 in the thin film 102 and separating each region of the plurality of nanopores 101 by a barrier wall. In the array device, the first liquid tank 104A can be a common tank, and the second liquid tank 1048 can be a plurality of individual tanks. In this case, the electrode can be disposed in each of the common tank and the plurality of individual tanks.

The biomolecule measurement device 100 includes an electrode pair 105. The electrode pair 105 includes a first electrode 105A and a second electrode 105B. The first electrode 105A is provided in the first liquid tank 104A. That is, for example, it is provided in contact with the first liquid tank 104A or inside the first liquid tank 104A. The second electrode 105B is provided in the second liquid tank 104B. That is, for example, it is provided in contact with the second liquid tank 104B or inside the second liquid tank 104B.

An electrolyte solution 103 is accommodated in the first liquid tank 104A and the second liquid tank 104E. As the electrolyte contained in the electrolyte solution 103, for example, KCl, NaCl, CsCl, or the like is used. As a buffer contained in the electrolyte solution 103, for example, Tris, EDTA, PBS, or the like is used. The first electrode 105A and the second electrode 105B can be formed of, for example, Ag, AgCl, Pt, Au, or the like.

A biomolecule 109 (DNA strand or the like) as a measurement target is introduced into the electrolyte solution 103. The biomolecule 109 includes a molecular motor 110 including, for example, a polymerase and a control chain 111 at one end thereof. Furthermore, the control chain 111 is bonded to a primer 112 at one end on the side far from the molecular motor 110, and has a spacer 113 at one end on the side close to the molecular motor 110. Due to the presence of the spacer 113, the primer 112 is not in contact with the molecular motor 110, and the synthesis reaction does not proceed until the biomolecule 109 reaches the inside of the nanopore 101. When the molecular motor 110 reaches the nanopore 101, deformation or the like occurs in the control chain 111, and the primer 112 comes into contact with the molecular motor 110. This initiates the synthesis reaction. That is, the synthesis start timing of the molecular motor 110 is controlled by the above structure.

The biomolecule measurement device 100 includes an ammeter 106 and a voltage source 107. The voltage source 107 applies a voltage between the first electrode 105A and the second electrode 105B. The ammeter 106 measures a current value flowing between the first electrode 105A and the second electrode 105B.

The bin molecule measurement device 100 includes a computer 108. The computer 108 has a configuration as a known computer, and includes, for example, an operation means and a storage means. The operation means includes, for example, a processor, and the storage means includes, for example, a storage medium such as a semiconductor memory device and a magnetic disk device. A part or all of the storage means may be a non-transitory storage medium.

Furthermore, the computer 108 may include an input/output device. The input/output device includes, for example, an input device such as a keyboard and a mouse, an output device such as a display and a printer, and a communication device such as a network interface.

The storage means may store a program. When the processor executes this program, the computer 108 may execute the functions described in this embodiment.

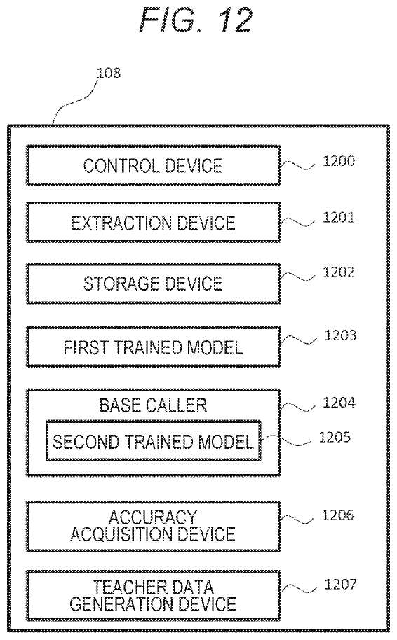

FIG. 12 illustrates a functional block diagram of the computer 108. The computer 108 includes a control device 1200, an extraction device 1201, a storage device 1202, a first trained model 1203, a base caller 1204, an accuracy acquisition device 1206, and a teacher data generation device 1207. The base caller 1204 includes a second trained model 1205. These functional units are realized, for example, by cooperation of the operation means and the storage means of the computer 108.

The computer 108 functions as the control device 1200, and can control voltages applied to the first electrode 105A and the second electrode 105B.

When a voltage is applied between the first electrode 105A and the second electrode 105B, a potential difference is generated between both surfaces of the thin film 102, and the biomolecules 109 dissolved in the first liquid tank 104A migrate in the direction of the second liquid tank 1048. The ammeter 106 includes an amplifier that amplifies a current value flowing between the electrodes by application of a voltage, and an analog to digital converter (ADC) (not illustrated). A detection value which is an output of the ADC is transmitted to the computer 108 as a current value. The computer 108 receives and stores the current value in the storage device 1202.

The signal indicating the measured current value is a blocking signal related to an event in which the biomolecule 109 blocks the nanopore 101. The computer 108 functions as the extraction device 1201, identifies a plurality of blocking events of the nanopore 101 based on the current value measured by the ammeter 106, and can extract a plurality or units of blocking event data representing these blocking events.

Each blocking event corresponds to, but is not limited to, an event in which one biomolecule 109 has blocked the nanopore 101. In addition, the blocking event data represents a blocking event of the nanopore 101 in the biomolecule measurement device 100, and can be data representing a current waveform as a specific example, but is not limited thereto. In addition, the data representing the current waveform may be, for example, data representing a time series of current values.

Note that the data representing the current waveform is riot limited to a numerical value of the measured current value as it is, and may represent the current waveform using a feature (average value or the like) to be described later. That is, the blocking event data may be data indicating the feature of the blocking event. If the feature is used in this way, there is a case where the classification accuracy or the blocking event data is improved as compared with a case where a numerical value obtained by quantifying the measured current value is used as it is.

For example, blocking event data obtained in association with an event that one biomolecule 109 has blocked the nanopore 101 can be interpreted as 1 unit of data. The blocking event data is one unit may include a plurality of information units (for example, time series data of current values).

An additional electrode may be provided in the nanopore 101. According to such a configuration, it is possible to acquire a tunnel current or detect a change in transistor characteristics, and it is possible to obtain information of the biomolecule 109 in more detail.

In addition, as described later, the computer 108 can acquire sequence information of the biomolecule 109 based on the blocking event data.

Note that in the biomolecule measurement device 100 described above, a part other than the computer 108 may be replaced with any known configuration.

FIG. 2 is a flowchart illustrating an example of a data processing method according to the present embodiment. When a voltage is applied to the electrode pair 105, a current according to the structure of the nanopore 101 and the electrical conductivity of the solution flows. When an event (blocking event) that the biomolecule 109 to be measured passes through the nanopore 101 occurs, a series of current values is detected as a signal (blocking signal) related to the blocking event (step 201). That is, the electric resistance value near the nanopore is temporally changed by the biomolecule, and the current value is temporally changed by the electric resistance value being changed. The computer 108 acquires and stores a signal representing this current value.

The computer 108 functions as the extraction device 1201, specifies a plurality of blocking events based on the current value measured by the ammeter 106, and extracts blocking event data related to each blocking event (step 202). The extracted blocking event data is stored in the storage device 1202 of the computer 108. The configuration and method for identifying the plurality of blocking events based on the time series data of current values can be optionally designed by a person skilled in the art. For example, a known technique may be used.

Here, among the blocking events, a blocking event that is not related to a biomolecule that is a measurement target is mixed. For example, the blocking event related to impurities does not relate to the measurement target. The blocking event to be extracted as a blocking event related to the measurement target is, for example, a blocking event related to a structure in which a control chain and a molecular motor are connected to an end portion of DNA, and bonded to a primer on the upstream side thereof. However, in practice, not only such DNA to which the molecular motor and the primer are connected, but also DNA to which the molecular motor is not connected and DNA to which the primer is not connected may be electrophoresed through the nanopore and observed as a blocking event.

In addition, even if the molecular motor is connected to DNA, the signal may become unstable due to a decrease in the activity of the molecular motor. Further, only a molecular motor (for example, polymerase or helicase) may cause a blocking event alone. It is also conceivable that other particles or impurities contained in the solution cause a blocking event.

As described above, there is a case where a blocking event that is not related to the measurement target is mixed as noise among the blocking events. In such a case, the analysis accuracy of the biomolecule may decrease. For example, a biomolecule that is not a measurement target may be erroneously recognized as a measurement target.

Therefore, it is effective to classify the blocking event data into data relating to the correct measurement target and data not relating to the correct measurement target, and analyze the biomolecule using only the good data. Hereinafter, the blocking event data related to the correct measurement target is referred to as “good data”, and the blocking event data that is not related to the correct measurement target is referred to as “bad data”.

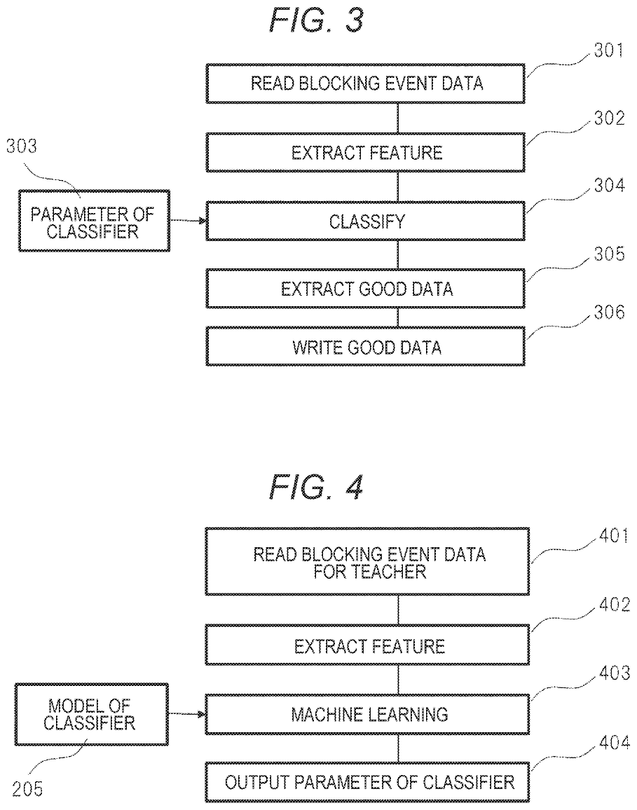

In the present embodiment, a trained model by machine learning is used. Specifically, a plurality of blocking event data is input to the first trained model 1203, and in response to this, the first trained model 1203 classifies each of the blocking event data into good data or bad data (step 203). As described above, in the present embodiment, the first trained model 1203 classifies the blocking event data representing the blocking event of the nanopore in the biomolecule measurement device. A specific operation in step 203 will be described later with reference to FIG. 3. A method for generating the first trained model 1203 (step 205) will be described later with reference to FIG. 4.

In addition, based on the blocking event data classified as good data, second trained model 1205 functions as a base caller and determines the base sequence of the biomolecule (step 204). A method for generating the second trained model 1205 (step 206) will be described later with reference to FIG. 4.

As an example of the second trained model 1205, a model obtained by optimizing a neural network by deep learning can be used. Specifically, after the parameters are optimized by deep learning using a network combining a convolution network, a recurrent neural network, and the like, the base sequence is decoded from the current waveform included in the blocking event data. Alternatively, the base sequence may be decoded by comparison with a current waveform measured using a dynamic time warping method (DTW). In any base call method, by extracting only the data related to the correct measurement target from the blocking event data and base calling in this manner, the base calling from data other than the measurement target does not occur, and highly accurate sequencing becomes possible.

FIG. 3 is a flowchart illustrating an example of a method for classifying blocking event data according to the Present embodiment. The computer 108 first reads the blocking event data (step 301). Next, the computer 108 extracts a feature of each blocking event data (step 302). As the feature, for the current value or its time series, one or more of an average value, a median value, a variance, a spectral center value, a spectral bandwidth, intensity of a specific frequency component, a zero crossing rate, a chromatogram, and a mel-frequency cepstrum coefficient can be used. In addition to or instead of these values, temporal changes in these values can be used. As a zero crossing rate, a value obtained by removing the DC component of the blocking event data can be used.

In addition, data obtained by discretizing information in the time axis direction and/or the current axis direction of the blocking event may be used as the feature. First, an example of discretization in the current axis direction will be described. Different discretized current values can be previously determined according to each type of base of the biomolecule. That is the current value represented by the blocking event data can take one of a plurality of discretized values. Each of the plurality of discretized values corresponds to one of the bases of the biomolecule. A specific example will be described later with reference to FIG. 11.

Next, an example of discretization in the time axis direction will be described. Among the biomolecules, the blocking current value varies depending on the base passing through the nanopore, but the rate of transporting the base by the molecular motor varies and is not constant. Therefore, the base transport speed, that is, the variation in the time axis direction may be corrected, and normalized data may be used. Specifically, the current waveform related to the blocking event data is corrected in the time direction and the current direction and further discretized according to the type of base transported by the molecular motor. The feature may be further calculated from the discretized current waveform.

By appropriately discretizing the data, the classification accuracy can be improved.

The computer 108 acquires parameters representing the first trained model 1203 constituting the classifier (step 303). The parameter is, for example, a set of weights of connections between neurons in the neural network. An example of a parameter generation method will be described later with reference to FIG. 4. The computer 108 configures the first trained model 1303 using this parameter. The computer 108 may execute step 305 in advance to configure the first trained model 1203.

The first trained model 1203 configured based on step 303 acquires the feature extracted in step 302 and classifies the blocking event data based thereon (step 304). As a result, good data is extracted (step 305) and output (step 306). The output destination is, for example, an output device of the computer 108, but may be a storage means (for example, the storage device 1202) of the computer 108 or another computer.

FIG. 4 is a flowchart illustrating an example of a training method for generating a first trained model 1203 constituting a classifier according to the present embodiment. The processing of FIG. 4 is executed by the computer 108 in the present embodiment, but may be executed by another computer as a modification.

In the present embodiment, the above-described first trained model 1203 is generated by executing machine learning of a training model using a plurality of units of teacher data (first teacher data). The first teacher data includes blocking event data (teacher blocking event data) and a label (teacher label).

The teacher blocking event data can be data in the same format as the blocking event data used in the processing of FIG. 3. For example, in a case where the blocking event data is data indicating the feature in the processing of FIG. 3, the teacher blocking event data is also data indicating the feature, and in a case where the blocking event data is discretized in the processing of FIG. 3, the teacher blocking event data is also discretized.

The teacher label represents whether the associated teacher blocking event data is classified as good data or bad data. The teacher blocking event data related to the correct measurement target is classified as good data, and the teacher blocking event data not related to the correct measurement target is classified as bad data.

Each label may be further subdivided. For example, the bad data may be further classified into those related to the blocking event by the molecular motor, those related to the blocking event of a biomolecule to which the molecular motor is not bonded, and the like.

The computer 108 reads the first teacher data (step 401). If the first teacher data does not directly represent the feature, the feature is extracted from the first teacher data (step 402). The machine learning is performed using this feature (step 403). As a result of the machine learning, a parameter representing the classifier (that is, the first trained model 1203) is output (step 404).

As described above, the machine learning of the training model is executed using the plurality of units of first teacher data, whereby the first trained model 1203 is generated. The generated first trained model 1203 will be configured to classify the blocking event data as good data or bad data, as described in connection with FIG. 3.

Although the processing for generating the first trained model 1203 has been described above, the second trained model 1205 can be similarly generated. Hereinafter, generation of the second trained model 1205 will be described, but description of points common to the first trained model 1203 may be omitted.

In the present embodiment, a second trained model 1205 is generated by executing machine learning of a training model using a plurality of units of teacher data (second teacher data). The second teacher data includes blocking event data (teacher blocking event data) and a base sequence (teacher base sequence). The teacher base sequence represents a correct base sequence related to the associated teacher blocking event data. Part or all of the teacher blocking event data included in the second teacher data may be the same as or different from the teacher blocking event data included in the first teacher data.

The computer 108 reads the second teacher data (step 401). If the second teacher data does not directly represent the feature, the feature is extracted from the second teacher data 402). The machine learning is performed using the feature (step 403), and a parameter is output (step 404).

As described above, the machine learning of the training model is executed using the plurality of units of second teacher data, whereby the second trained model 1205 is generated. The generated second trained model 1205 is used to determine the base sequence of the biomolecule based on the blocking event data, as described in connection with FIG. 2.

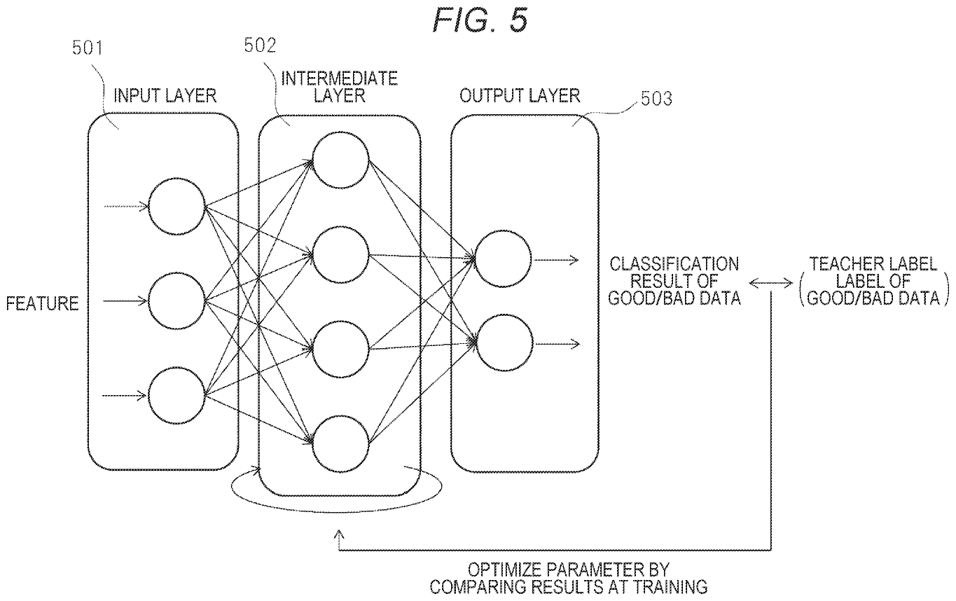

FIG. 5 is a diagram schematically illustrating an example of a training model according to the present embodiment and an example of machine learning processing thereof. Although generation of the first trained model 1203 ill be described below, generation of the second trained model 1205 can be similarly performed in this example, the training model includes a neural network.

The feature extracted from the blocking event data is input to an input layer. Each parameter of the input layer is weighted and connected to an intermediate layer. After a plurality of the intermediate layers, an output layer is connected. A label indicating a classification result is output from the output layer.

The output classification result is compared with the classification result represented by the teacher label of the first teacher data, and the weighting parameter of the classifier is optimized. The machine learning optimizes classifier parameters so that blocking event data can be classified into good data and bad data. The parameters of the finally optimized classifier are stored in a storage means (for example, the storage device 1202) of the computer 108, a database of another computer, or the like.

As described above, by using the first trained model 1203 optimized by the neural network as a classifier, the blocking event data can be classified and the blocking event data related to the correct measurement target can be extracted, so that highly accurate sequencing can be performed.

In FIG. 5, the configuration using the neural network has been described as the machine learning method, but the machine learning method is not limited thereto. A classification method using a support vector machine or the like may be used. Alternatively, a classification method such as nearest neighbor or simple Bayes may be used.

In addition, the above-described classification method may be combined with other methods. Specifically, a hierarchical classification method may be combined, or an unsupervised classification method (clustering) or the like may be combined.

Note that, at the time of the machine learning, it is possible to further increase the accuracy by adjusting so that false positives (bad data is mistaken as good data) are less than false negatives (good data is mistaken as bad data).

Depending on the measurement target, the blocking time may vary. In such a case, it is preferable to divide a long-time blocking event among the blocking events into a plurality of units of blocking event data by temporally dividing the blocking event.

In the first embodiment described above, the base call (step 204) is executed using the second trained model 1205, but as a modification, the base call may be performed by a known technique.

### Second Embodiment

A biomolecule measurement device according to a second embodiment of the present invention will be described below. In the second embodiment, input/output in the storage means (for example, the storage device 1202) of the computer in the first embodiment is particularly clarified. Hereinafter, description of parts common to the first embodiment may be omitted.

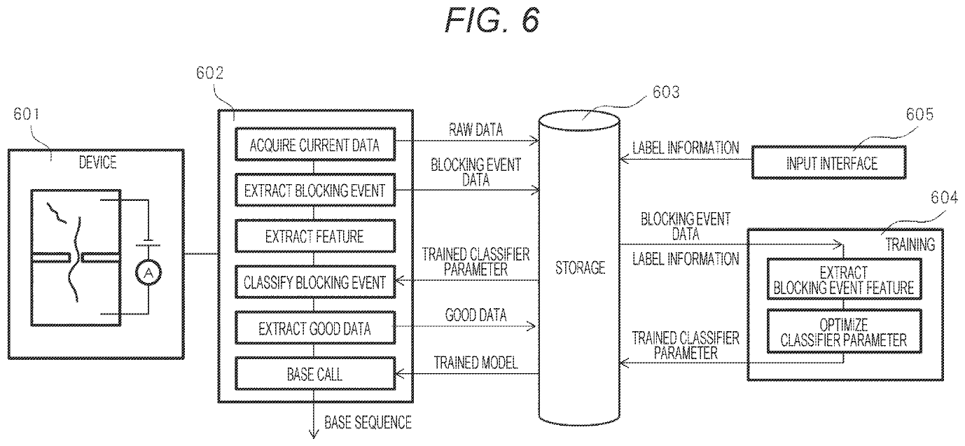

FIG. 6 is a diagram schematically illustrating a biomolecule measurement device according to the present embodiment. The biomolecule measurement device includes a nanopore current measurement device 601, a control unit 602, a storage 603, a training model 604, and an input interface 605. The control unit 602, the storage 603, the training model 604, and the input interface 605 may be configured by a single computer.

The nanopore current measurement device 601 is, for example, a portion of the first embodiment (FIG. 1) excluding the computer 108. The control unit 602 is, for example, an operation means of the computer 108, the storage 603 is, for example, a storage means (for example, the storage device 1202) of the computer 108, and the input interface 605 is, for example, an input device of the computer 108.

The training model 604 is used to generate the first trained model 1203, but is also applicable to the second trained model 1205. Note that, as in the first embodiment, a modification not using the second trained model 1205 is also possible.

Data acquired by the nanopore current measurement device 601 is taken into the control unit 602 as current data. The current data is stored in the storage 603. In addition, a blocking event that is a current waveform while the nanopore is blocked is extracted from the current data. The extracted blocking event data is stored in the storage 603.

A feature is extracted from the blocking event data. The blocking event data is classified by the first trained model 1203 using the extracted feature. A base call is made based on the blocking event data classified as good data, and a base sequence is output.

The first teacher data (and the second teacher data if necessary) can be input via the input interface 605. The optimized trained parameters are stored in the storage 603 and used to generate each trained model.

Note that the storage of data (current waveform data, blocking event data, and the like) in the storage 603 may be temporary, or the data may be discarded after necessary processing is completed. The hardware constituting the storage 603 may be in any form such as an HDD, an SSD, and a volatile memory.

In this way, it is possible to accurately determine the base sequence by extracting good data by machine learning and base calling.

### Third Embodiment

A biomolecule measurement device according to a third embodiment of the present invention will be described below. In the third embodiment, the result of the output by the second trained model 1205 in the second embodiment is fed back to the generation processing of the first trained model 1203. Hereinafter, description of parts common to the first or embodiment may be omitted.

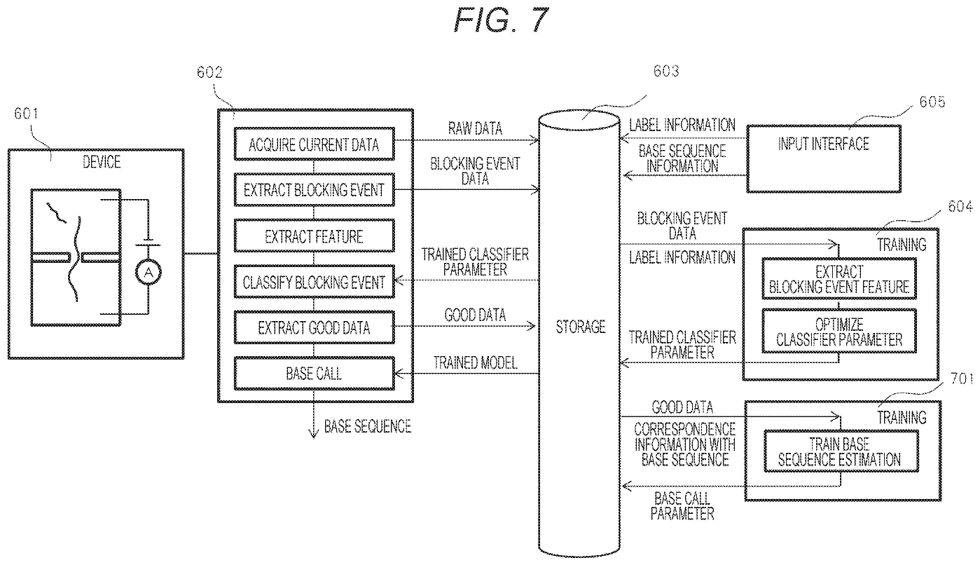

FIG. 7 is a diagram schematically illustrating a biomolecule measurement device according to the present embodiment. The biomolecule measurement device includes a trained model 701 for generating the second trained mode 1205 in addition to the training model 604 for generating the first trained model 1203.

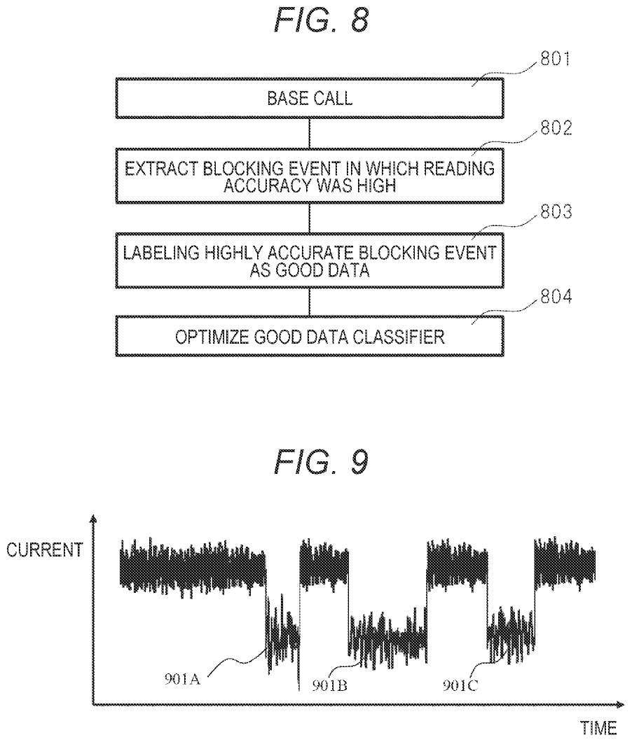

FIG. 8 is a flowchart illustrating an example of a feedback method according to the present embodiment. The processing of FIG. 8 can be executed by the computer 108 of the first embodiment, for example. First, the second trained model 1205 makes a base call (step 801). This step 801 corresponds, for example, to step 204 in the first embodiment (FIG. 2).

The computer 108 functions as the accuracy acquisition device 1206 to evaluate the accuracy of the base call result and classify it into blocking event data whose accuracy satisfies a predetermined criterion and blocking event data whose accuracy does not satisfy a predetermined criterion (step 802). For example, one with high accuracy is extracted. The accuracy of the base call is represented, for example, by the accuracy of the base sequence, and can be calculated for each blocking event data (or for each biomolecule). As a specific example, a value obtained by dividing the number of bases correctly decoded in the base sequence of the biomolecule by the total number of bases contained in the base sequence can be used as the accuracy. Whether or not the accuracy is high can be determined by comparison with a predetermined threshold. In this way, for the base sequence determined in step 801, accuracy is obtained in step 802.

The computer 108 functions as the teacher data generation device 1207, and generates first teacher data by adding an appropriate teacher label to each base sequence if the accuracy satisfies a predetermined criterion (for example, if the accuracy is high) (step 803). For example, teacher blocking event data is generated based on the blocking event data related to the base sequence, and a teacher label indicating good data is added to the teacher blocking event data to obtain first teacher data. Similarly, for each base sequence, in a case where the accuracy does not satisfy a predetermined criterion (for example, in a case where the accuracy is not high), the teacher blocking event data is generated based on the blocking event data related to the base sequence, and a teacher label indicating bad data may be added to the teacher blocking event data to obtain first teacher data.

The first teacher data generated in this way can be used for generation processing of the first trained model 1203 illustrated in FIG. 4. In this way, it is possible to perform the machine learning in consideration of not only whether or not the blocking event data relates to the correct measurement target but also whether or not the base sequence can be correctly decoded, so that the decoding accuracy of the base sequence is further improved.

### Fourth Embodiment

A biomolecule measurement device according to a fourth embodiment of the present invention will be described below. The fourth embodiment specifically illustrates an example of a current waveform in any of the first to third embodiments. Hereinafter, description of parts common to any of the first to third embodiments may be omitted.

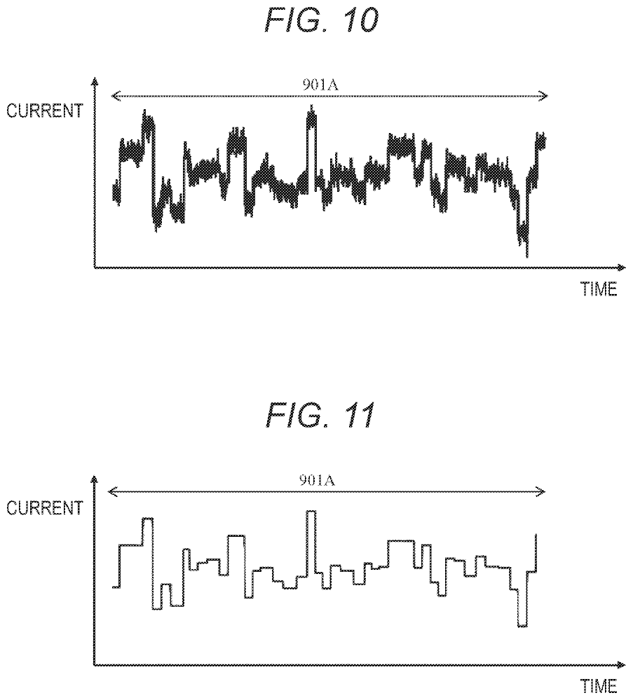

FIG. 9 illustrates an example of a current waveform according to the fourth embodiment. The current waveforms include blocking event data 901A, 901B, and 901C. FIG. 10 illustrates an enlarged view of the blocking event data 901A. FIG. 11 illustrates a discretized blocking event data 901A.

In FIG. 11, the current level is discretized according to the level corresponding to each base of the biomolecule as the measurement target, and the noise included in FIG. 10 is reduced. In this manner, the influence of noise can be suppressed by discretization, and the classification accuracy can be improved.

### Reference Signs List

100 biomolecule measurement device
101 nanopore
102 thin film
103 electrolyte solution
104 liquid tank (104A first liquid tank, 104E second liquid tank)
105 electrode pair (105A first electrode, 105B second electrode)
106 ammeter
107 voltage source
108 computer
109 bio molecule
110 molecular motor
111 control chain
112 primer
113 spacer
601 nanopore current measurement device
602 control unit
603 storage
604 training model
605 input interface
701 training model
901A to 901C blocking event data
1200 control device
1201 extraction device
1202 storage device
1203 first trained model
1204 base caller
1205 second trained model
1206 accuracy acquisition device
1207 teacher data generation device

1. A method for generating a trained model for classifying blocking event data representing a nanopore blocking event in a biomolecule measurement device, the method comprising:
generating a first trained model by executing machine learning of a training model using first teacher data, wherein
the first teacher data includes teacher blocking event data and a teacher label, and the teacher label indicates whether the teacher blocking event data is classified as good data or bad data, and
the first trained model is configured to classify the blocking event data into good data or bad data.

2. A method for determining a base sequence of a biomolecule, the method comprising:
inputting blocking event data representing a blocking event of a nanopore in a biomolecule measurement device to a first trained model generated using the method according to claim 1;
classifying the blocking event data into good data or bad data by the first trained model; and
determining a base sequence of a biomolecule based on the blocking event data classified as good data.

3. The method according to claim 1, wherein the blocking event data and the teacher blocking event data are data representing a feature of the blocking event.

4. The method according to claim 2, wherein the blocking event data and the teacher blocking event data represent respective current values, and the current values can take respective ones of a plurality of discretized values, and
each of the plurality of discretized values corresponds to one of the bases of the biomolecule.

5. The method according to claim 2, wherein
the base sequence is determined based on the blocking event data by using a second trained model,
the second trained model is generated by executing machine learning of a training model using second teacher data, and
the second teacher data includes teacher blocking event data and a teacher base sequence.

6. The method according to claim 5, further comprising:
acquiring accuracy for the determined base sequence; and
generating the teacher blocking event data related to the good data based on the blocking event data related to the base sequence if the accuracy satisfies a predetermined criterion.

7. The method according to claim 1, wherein the training model includes a neural network.

8. A biomolecule measurement device comprising:
a first liquid tank;
a second liquid tank;
a thin film on which nanopores are formed, the thin film being disposed between the first liquid tank and the second liquid tank;
a first electrode provided in the first liquid tank;
a second electrode provided in the second liquid tank;
an ammeter that measures a current value flowing between the first electrode and the second electrode;
an extraction device that extracts blocking event data based on the current value measured by the ammeter;
a storage device that stores the blocking event data;
the first trained mode according to claim 1 that classifies the blocking event data into good data or had data; and
a base caller that determines a base sequence of a biomolecule based on the blocking event data classified as the good data.

9. The biomolecule measurement device according to claim 8, wherein the thin film is formed of a solid material, and the nanopore is a pore penetrating the solid material.

10. The biomolecule measurement device according to claim 8, wherein the blocking event data and the teacher blocking event data are data representing a feature of the blocking event.

11. The biomolecule measurement device according to claim 8, wherein the current value can take one of a plurality of discretized values, and
each of the plurality of discretized values corresponds to one of the bases of the biomolecule.

12. The biomolecule measurement device according to claim 8, wherein
the base caller includes a second trained model, the second trained model is generated by executing machine learning of a training model using second teacher data, and
the second teacher data includes teacher blocking event data and a teacher base sequence.

13. The biomolecule measurement device according to claim 8, further comprising:
an accuracy acquisition device that acquires accuracy for the determined base sequence; and
a teacher data generation device that generates the teacher blocking event data related to the good data based on the blocking event data related to the base sequence if the accuracy satisfies a predetermined criterion.

14. The biomolecule measurement device according to claim 8, wherein the training model includes a neural network.
